(19) United States
(12) Patent Application Publication
(10) Pub. No.: US 2002/0151873 A1
(43) Pub. Date: Oct. 17, 2002

(54) Laparoscopic anesthetic sprayer system

(76) Inventor: Mark Moore, Tallahassee, FL (US)

Correspondence Address: Innovation Park, 1673 W. Paul Dirac Drive, Tallahassee, FL, 32310-3763, US

(21) Appl. No.: 09/836,522

(22) Filed: Apr. 17, 2001

(51) Int. Cl. A61M 25/16

(52) U.S. Cl. 604/537; 604/48

(57) ABSTRACT

The present invention is a tool for use with laparoscopic surgery. The tool includes a hollow member having a first end and a second end. Each end is open and the first end is adapted to receive a conventional vile or syringe housing anesthetic medicine. The second end includes a sprayer head for enabling the housed anesthetic medicine to be dispensed via the sprayer head.

### BACKGROUND OF THE INVENTION

1. Field of the Invention

The present invention relates generally to a laparoscopic anesthetic sprayer system for use in a laparoscopic surgical case and more particularly to a laparoscopic anesthetic sprayer system having a unique sprayer head that allows physicians to adequately and efficiently inject a specific dose of local anesthetic over a specified time period to internal membranes where it is easily absorbed during laparoscopic surgeries and to inherently reduce post operative pain.

2. Description of the Prior Art

Local anesthetics are used in the majority of open surgical cases. The patient benefits of local anesthetics in open surgical cases include a reduction in post-operative pain, smoother emergence from the surgery, lower narcotic needs, earlier discharge, less cardiovascular volatitlity, and less nausea and vomiting.

As such, it is seen that currently there is no effective or efficient manner to inject local anesthetics during laparoscopic surgeries. The present invention allows the surgeon to give the same quality of care and benefits to their laparoscopic cases as to their open surgical patients.

For example as seen in U.S. Pat. No. 5,605,545 issued to Nowosielski et al wherein disclosed is a system that provides for continuous or intermittent fluid delivery to the internal surgical site during certain surgical procedures such as urological, gynecological, and laparoscopic surgeries. The system includes a fluid source, a flexible tube with one end connected to a fluid source and the other end connected to a surgical instrument, and a peristaltic type pump. Although this device may provide a fluid source during certain surgical procedures, it will not deliver local anesthesia.

With the advance of technology, the use of laparoscopic operative procedure, whereby the surgeon can work on the inside of the body with instruments through tiny ports inserted through the skin, is replacing many traditional “open” surgeries. An advantage of laparoscopic operative procedures is that it is less traumatic to the patient and allows for quicker recovery time. In addition, laparoscopic procedures have less morbidity and mortality. However, although the medical technology of laparoscopic operative procedures has advanced, the procedure has some major limitations. Currently, there is no effective or efficient manner to inject local anesthetics during laparoscopic surgeries.

As observed, none of the previous inventions as described above have been utilized by the medical community to assist surgeons in effectively injecting local anesthetics during laparoscopic operative surgeries. Thus it is seen that these previous efforts do not provide the benefits intended with the present invention or method, such as providing a laparoscopic anesthetic sprayer system that will allow surgeons to provide the same quality of care and benefit to laparoscopic surgical patients as their open surgical patients by delivering local anesthetics to laparoscopic surgical sites. The present invention provides a laparoscopic anesthetic spray system to be inserted through the same ports already in place for the procedure, which delivers a specific dose of local anesthetics in an efficient and effective manner. In addition, the system of the present invention provides for an adjustable spray head which can be easily and efficiently positioned to accurately deliver local anesthetics to the internal surgical site, and consequently provides a cost effective system that would be easily adaptable and successfully used by the medical community. Additionally, prior techniques do not suggest the present inventive combination of component elements as disclosed and claimed herein.

Accordingly, it is seen that there is a need for a laparoscopic anesthetic sprayer system with an adjustable distributor head designed and configured to offer the most advanced technology available today to the medical community.

Thus, as will be seen, the present invention achieves its intended purposes, objectives and advantages over the prior art devices by accomplishing the needs and objectives as identified herein, through a new, useful and unobvious combination of component elements, which is simple to use, with the utilization of a minimum number of functioning parts, at a reasonable cost to manufacture, assemble, test and by employing only readily available material.

### SUMMARY OF THE INVENTION

The present invention is a laparoscopic anesthetic sprayer system ideally suited for injecting a specified dose of local anesthetic over a specific time period to internal surgical sites. During open laparoscopic surgery, local anesthetic is delivered to the particular site as a means of reducing post operative pain for inherently providing smoother emergence, lower narcotic needs, and optimally earlier discharge. Utilizing the present invention will also innately reduce intraoperative general anesthetic needs for faster emergence and awaking while lowering the use of expensive antimetics. Providing a means of earlier discharge and quicker recovery will consequently provide a unit that will lower nursing care workload as well as lower costs to hospitals and insurers.

In order to provide for such benefits, the present invention includes a hollow tubular member having a first end and a second end. The second end is designed to receive a conventional syringe. This conventional syringe will house the specific dose of local anesthetic that is typically used in particular laparoscopic procedure.

The second end includes an adjustable sprayer head that will adequately and efficient dispense an anesthetic fluid. To render adequate distribution properties, the adjustable head includes a surface area that includes a plurality of apertures. As anesthetic fluid enters the first end of the hollow tubular member and travels to the second end, the anesthetic fluid is able to be distributed over a large and broad area so as to effectively dispense the particular anesthetic fluid that is housed in the syringe.

In the preferred embodiment, to enhance the product of the present invention, the head can be pivotally secured to the elongated tube, so as to enable the head to rotate about the second end and permit the user to adjust and control the path of the anesthetic fluid. This pivotal attachment will provide for the flow or spray of anesthetic to occur frontward, sideways and the like. Optionally, the head can further include an adjustment device so as to control and adjust the pattern of the anesthetic fluid. This will provide for the user to control the flow pattern of the particular anesthetic fluid housed within the syringe. In another embodiment, the sprayer head may be stationary.

Utilizing the present invention occurs when the user adjusts the sprayer head to the desired position. When at the desired position, during surgery or the like, the user inserts the second end of the hollow tubular member into the existing portal of the patient. Once inserted, the conventional syringe is placed therein to enable the anesthetic fluid housed to be dispensed in the desired area. As the user presses on the conventional syringe, the anesthetic fluid travels through the hollow tube to the second end and exists the sprayer head. After all the fluid has exited, the present invention is removed. The particular medical procedure can then continue as directed by the attending physician.

The present invention will enable the patient to receive a standard dose of local anesthetic over a specific time period during laparoscopic surgery in an efficient and effective manner. The instant invention delivers this specific anesthetic dose to a broad area of the internal body membranes where they are easily absorbed and also reduce postoperative pain.

Using and operating the present invention is simple, effective and extremely efficient. The present invention will elevate the role of laparoscopic operative procedures in modern medicine. In addition, the novel and unique laparoscopic anesthetic sprayer system will greatly advance the technology in the art of laparoscopic local anesthetic operative procedures.

Accordingly, it is the primary object of the present invention to provide for a laparoscopic anesthetic sprayer system to be used by surgeons with the capability of enabling the surgical team to deliver a standard dose of local anesthetic in an efficient and effective manner during laparoscopic procedures.

A further object of the present invention is to provide for laparoscopic anesthetic sprayer system, which will deliver a dose of local anesthetics over a specific time period to a broad area of internal laparoscopic surgical field of the patient.

Another object of the present invention is to provide for a laparoscopic anesthetic sprayer system which is simple in design and one that can be utilized with a minimal amount of training.

Yet another object of the present invention is to allow surgeons and the surgical team to give laparoscopic surgical patients the same advantages afforded open surgical cases such as reduced pain, smoother emergence from surgery, lower nausea and/or vomiting, potentially lower morbidity and mortality, as well as faster hospital discharge with attractive cost benefit ratios.

A further object of the present invention is to provide for a laparoscopic sprayer system that will overcome the deficiencies, shortcomings, and drawbacks of the prior laparoscopic devices and methods thereof.

A final object of the present invention to be specifically enumerated herein, is to provide a laparoscopic anesthetic sprayer system in accordance with the preceding objects and which will conform to conventional forms of manufacture, be of simple construction and easy to use so as to provide a system that would be economically feasible, long lasting and relatively trouble free in operation.

The present invention meets the requirements of a simplified design, compact size, low initial cost, low operating cost, ease of installation and maintainability, and minimal amount of training to successfully employ the invention.

The foregoing has outlined some of the pertinent objects of the present invention. These objects should be construed to be merely illustrative of some of the more prominent features and application of the intended invention. Many other beneficial results can be obtained by applying the disclosed invention in a different manner by modifying the invention within the scope of the disclosure. Accordingly, a fuller understanding of the invention may be had by referring to the detailed description of the preferred embodiment, in addition to the scope of the invention taken in conjunction with the accompanying drawings.

### BRIEF DESCRIPTION OF THE DRAWING

FIG. 1 is a rear perspective view of one embodiment of the laparoscopic anesthetic sprayer of the present invention.

FIG. 2 is front side view of the laparoscopic anesthetic sprayer, as illustrated in FIG. 1, of the present invention.

FIG. 3 is a cross-sectional view of the laparoscopic anesthetic sprayer, as illustrated in FIG. 1 of the present invention.

FIG. 4 is an enlarged partial cut-away view of the top end of the laparoscopic anesthetic sprayer of the present invention.

FIG. 5 is a side perspective view of an alternative embodiment for the laparoscopic anesthetic sprayer of the present invention.

FIG. 6 is cross-sectional view of the laparoscopic anesthetic sprayer as illustrated in FIG. 5, of the present invention.

Similar reference numerals refer to similar parts throughout the several views of the drawings.

### DESCRIPTION OF THE PREFERRED EMBODIMENT

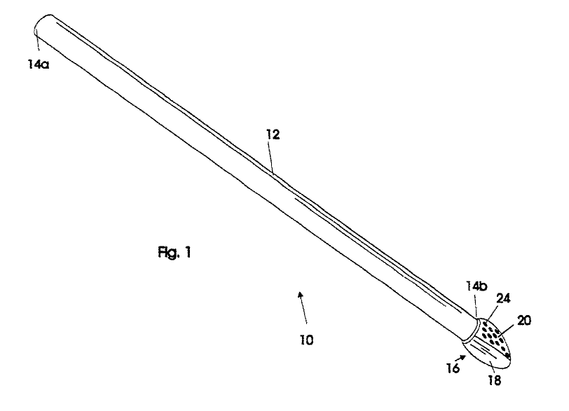

As seen in the drawings, in particular to FIGS. 1-6 there is shown a laparoscopic anesthetic sprayer, denoted by reference numeral 10, that is designed and configured to overcome the deficiencies generally associated with conventional laparoscopic devices, such as providing a unit that will adequately inject an anesthetic fluid to a particular area of a patient. The laparoscopic anesthetic sprayer is ideally suited for use in laparoscopic surgical procedures so as to provide for the present invention to be inserted into the existing portal of the patient.

In order to achieve such a configuration, the laparoscopic anesthetic sprayer 10 of the present invention, as seen in FIGS. 1-3, comprises an elongated hollow member 12 having a first end or upper end 14a and a second end or lower end 14b. The first end or upper end 14a and the second end or lower end are open. This will enable the first end to accept or receive a vile containing anesthetic medicine or optionally, to receive a syringe, as seen in FIG. 4. Thus providing for the first end or upper end 14a to act as an inlet.

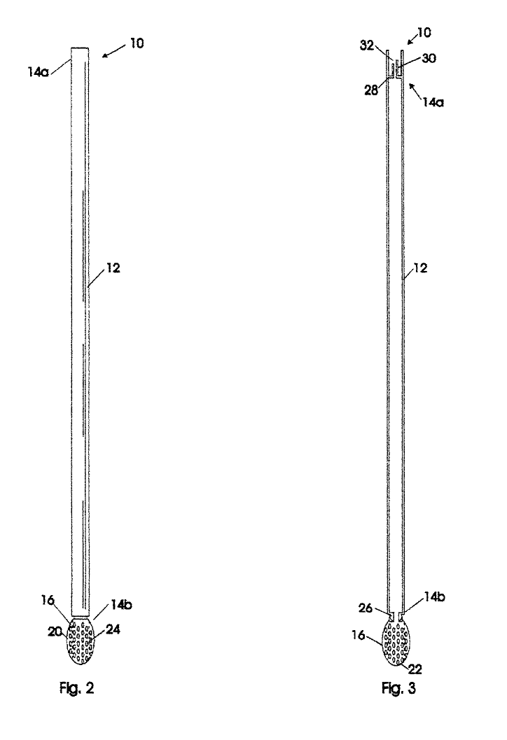

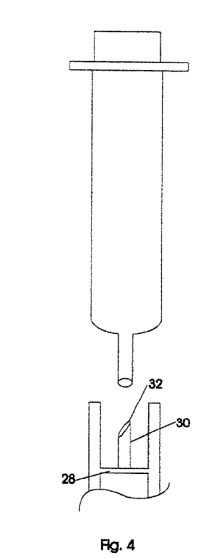

Secured to second end or lower end 14b, as seen in FIGS. 1-3 is a sprayer head 16. This sprayer head includes a flat planar back wall 18. Extending upward from the outer edges of the back wall are sidewalls 20. Coupling the sidewalls is a front wall 22. As seen in FIGS. 2 and 3, a dash line is used to illustrate the division between the front wall and sidewalls. This configuration is ideal for inserting the spray head into the existing portal. The flat planar back provides for a tool that is non-obtrusive while the curved walls provide for a head that will not be obtrusive to the vital areas of the body when the device is inserted into the existing portal. Extending though the front wall of the spray head, and optionally through the sidewalls of the spray head are a plurality of apertures 24.

In the preferred embodiment, to aid in the use of use of the present invention, the sprayer head 16 can be attached to the second end 14b of the hollow member 12 via a conventional ball and socket unit. This will provide for the sprayer head to be pivotally secured to the second end of the hollow member 12. As seen in FIG. 3, the conventional ball and socket unit provides for the second end 14b to include a ball member 26 having a channel extending therethrough. The ball member 26 is received within a socket member and the socket member includes an open end. Once anesthetic fluid enters into the hollow member 12, it travels to the second end. The fluid can then travel from the second end through the channel and exit via the open end of the socket though the apertures that extend the spray head. Thus providing for the apertures 24 to act as an outlet.

In another embodiment, (not shown) the sprayer head 16 can be attached to the second end 14b of the hollow member 12 in a stationary fashion.

The first end of the hollow member will receive a vile of anesthetic medicine or a conventional syringe. In order to receive the conventional elements, the upper end provides for a seat portion 28, as seen in FIGS. 3 and 4. The seat acts as a stop or rest and prevents the vile or syringe (illustrated in FIG. 4 but not labeled) from entering into the hollow member. Extending upward from the seat portion 28 is a hollow tubular member 30. This hollow tubular member 30 guides the anesthetic liquid/medicine housed within the vile or syringe to the hollow member 12. In the preferred embodiment, as seen in the drawings, the hollow tubular member 30 includes a tip 32. This tip will aid in piercing the protective seal generally found on a vile of medicine. Thus providing a means of opening the housed substance.

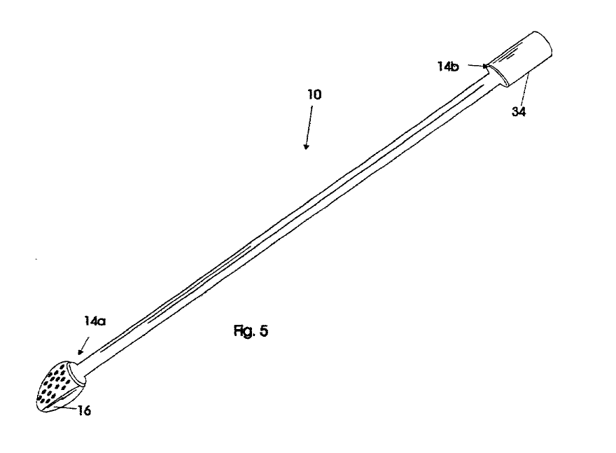

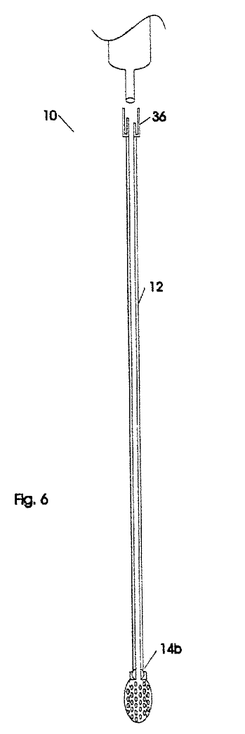

The hollow member 12 can be decreased in diameter as seen in FIGS. 5 and 6. This will provide for an alternative configuration of the present invention. As seen in these figures, decreasing the hollow member 12 will provide for the first end 14a to be enlarged so as to provide for the enlarged end to act as a holding receptacle for the vile or syringe, as seen. The internal mechanism of the first and second ends remains the same. Thereby providing for the first end to include a seat having a hollow tubular member extending upward therefrom. Outer walls 32 of the enlarged end are ideal for support the tip of the syringe or vile, so as to snuggly maintain the container in a fixed and secure position. The second end can optionally contain a conventional ball and socket configuration for rendering the sprayer 16 to be pivotally secured to the hollow member 12.

In use, the attending physician, if provided, pivots the sprayer 16 to the desire location. Once at the desired location, the sprayer is inserted into the existing portal of the patient. The anesthetic spray is placed over the area to be sprayed with the particular medicine. The anesthetic medicine is secured to the top end. Once secured, disbursement of the anesthetic occurs. During open laparoscopic surgery, the present invention will enable local anesthetic to be delivered to the particular site as a means of reducing post operative pain for inherently providing smoother emergence, lower narcotic needs, and optimally earlier discharge. Utilizing the present invention will also innately reduce intraoperative general anesthetic needs for faster emergence and awaking while lowering the use of expensive antimetics. Providing a means of earlier discharge and quicker recovery will consequently provide a unit that will lower nursing care workload as well as lower costs to hospitals and insurers.

While the present invention has been particularly shown as embodiment thereof, it will be understood by those skilled in the art that various changes in form and detail may be the invention made in the invention without departing from the spirit and scope of present invention.

I claim:

1. A laparoscopic tool comprising:
a hollow elongated body member having a first end and a second end;
a receiving device is secured to said first end for receiving conventional anesthetic medicine;
a sprayer head is secured to said second end for dispensing said medicine; and
said sprayer head includes a plurality of apertures.

2. A laparoscopic tool as in claim 1 wherein said sprayer head is pivotally secured to said second end.

3. A laparoscopic tool as in claim 1 wherein said sprayer head includes a rear wall, side walls and front wall, said rear wall is flat and said side walls extend upwardly from outer edges of said rear wall, and said front wall couples said side walls.

4. A laparoscopic tool as in claim 3 wherein said plurality of apertures extend through said front wall.

5. A laparoscopic tool as in claim 3 wherein said plurality of apertures extends through said front wall and said sidewall.

6. A laparoscopic tool as in claim 1 wherein said second end is open and said second end includes a seat internally located therein, extending upwardly from said seat is a channel for receiving said conventional anesthetic medicine.

7. A laparoscopic tool as in claim 6 wherein said channel includes a pointed tip.

8. A laparoscopic tool as in claim 1 wherein said first end is open and enlarged.

9. A laparoscopic tool as in claim 2 wherein said pivotal attachment occurs via a ball and socket configuration.
